(19) United States
(12) Patent Application Publication
(10) Pub. No.: US 2009/0024415 A1
(43) Pub. Date: Jan. 22, 2009

(54) DEVICE AND METHOD FOR MEDICAL FACILITY BIOMETRIC PATIENT INTAKE AND PHYSIOLOGICAL MEASUREMENTS

(76) Inventors: Alan I. Alpert, Boca Raton, FL (US); Dawn E. Alpert, Boca Raton, FL (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/972,120

(22) Filed: Jan. 10, 2008

Related U.S. Application Data
(60) Provisional application No. 60/959,600, filed on Jul. 16, 2007.

(51) Int. Cl. G06Q 50/00; A61B 5/02

(52) U.S. Cl. 705/3; 705/2; 600/485; 600/300

(57) ABSTRACT

A biometric check-in and physiological measurement method and device provides a more convenient and comfortable check-in and physiological measurement process for the patient. With a scan of a part of the patient's body, the patient can be checked-in as well as have his blood pressure, body temperature, and heart rate measured. This method and device also reduces the number of receptionist, nurses, and exam rooms needed in a medical facility, thereby reducing staffing and facility costs.

### CROSS REFERENCE TO RELATED APPLICATION

This application claims the benefit of U.S. Provisional Application No. 60/959,600, filed Jul. 16, 2007, herein incorporated by reference.

### FIELD OF THE INVENTION

The present invention relates generally to patient intake procedures employing means for personal identification and identity authentication using biometric information, and advanced physiological measurement techniques.

### BACKGROUND

A conventional doctor's appointment, or more generally an appointment with a medical professional, involves a lot of time and resources spent by both the patient and the medical facility staff beyond that spent during the actual contact between the patient and the medical professional. Beyond medical professionals, which includes doctors and nurse practitioners, a patient typically interacts with a receptionist during the medical facility patient intake process. The intake process typically consists of patient registration, which occurs during a patient's initial visit to see a medical professional, and patient check-in, which occurs during a patient's subsequent visits. A medical facility includes, but is not limited to a hospital and doctor's office.

The intake process is typically followed by a series of physiological measurements, including blood pressure, heart rate, temperature, and weight measurements, on the patient by a medical staff, which includes a nurse and physician's assistant. Only after a lengthy patient intake and physiological measurement process, involving receptionists and medical staff, does a patient finally meet the medical professional. Not only is this patient intake and physiological measurement process costly and wasteful in terms of patients' and staffs' time and resources, there are also other inherent problems.

During the check-in procedure, a patient experiences the risk of compromising his personal information when the patient communicates that personal information to the receptionist. This personal information may include a patient's Social Security number, date of birth, and residence address. An eavesdropper may easily obtain this personal information for fraudulent purposes. During the check-in procedure, a patient who does not speak English typically needs to be accompanied by a translator. This limits the independence of patients who do not speak English.

There are also deficiencies with the conventional method of taking physiological measurements, after a patient has completed the intake process. These deficiencies are especially numerous with the conventional methods for taking blood pressure measurements, which includes a manual and an automated method. In a manual method, a medical staff member manually inflates a cuff placed around the patient's arm to occlude blood flow in an artery. While gradually deflating the cuff, the medical staff member uses a stethoscope to detect the presence or absence of a pulse in the artery, thereby determining the systolic pressure, the peak blood pressure in the artery, and the diastolic pressure, the minimum blood pressure in the artery. Deficiencies associated with the manual method for taking blood pressure measurements include human errors due to distractions, hearing loss, and lack of training. Inaccuracies may also arise from patient anxiety due to discomfort of the procedure, unfamiliarity with the medical staff member, and anticipation of high blood pressure readings.

In an automated method, an instrument automatically inflates the cuff and measures blood pressure, eliminating the need for a medical staff member to administer the procedure. However, new problems arise such as a patient improperly positioning the cuff, and inaccuracies due to patient anxiety still remain.

Besides blood pressure measurements, a medical staff member typically measures the patient's body temperature, weight, and heart rate. As these measurements are typically performed serially, each lasting a minute or two, the entire physiological measurement process is a time-consuming process for both the patient and the medical staff. There is the need for improvement in both the medical facility patient intake and physiological measurement processes.

### SUMMARY OF THE INVENTION

According to one aspect of the present invention, there is provided a biometric check-in and physiological measurement method for a patient arriving at a medical facility and interacting with a biometric check-in and physiological measurement device. The device identifies the patient or authenticates an identity of the patient using biometric information acquired from the patient, measures a physiological characteristic of the patient, and performs at least one of recording an arrival of the patient at the medical facility or verifying the patient has an appointment.

According to another aspect of the present invention, there is provided a biometric check-in and physiological measurement device, including means for identifying a patient or authenticating an identity of the patient using biometric information acquired from the patient, means for recording an arrival of the patient at a medical facility or verifying the patient has an appointment, and means for measuring a physiological characteristic of the patient.

According to another aspect of the present invention, there is provided a method, comprising a physiological measurement device measuring a physiological characteristic of a patient without a medical staff member's assistance and informing the patient or a medical personnel of a status of the patient.

According to another aspect of the present invention, there is provided a physiological measurement device, comprising means for measuring a physiological characteristic of the patient without a medical staff member's assistance and means for informing the patient or a medical personnel of a status of the patient.

Other aspects of the invention are described below and in connection with the description of the figures that follow.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1A shows a flow diagram of a conventional patient registration process;

FIG. 1B shows a flow diagram of a conventional patient check-in process;

FIG. 1C shows a flow diagram of a conventional physiological measurement process;

FIG. 2A shows a flow diagram of a patient biometric registration process, according to one embodiment of the invention;

FIG. 2B shows a flow diagram of a patient biometric check-in and physiological measurement process, according to one embodiment of the invention;

FIG. 2C shows a flow diagram of an alternate subroutine of the biometric check-in and physiological measurement process, according to one embodiment of the invention;

FIG. 2D shows a flow diagram of a self-serve physiological measurement process, according to one embodiment of the invention;

FIG. 3A shows a block diagram of a medical facility configured for biometric registration, biometric check-in, and physiological measurement, according to one embodiment of the invention;

FIG. 3B shows a block diagram of a medical facility configured for biometric registration, biometric check-in, and physiological measurement, according to one embodiment of the invention;

FIG. 3C shows a block diagram of a medical facility configured with a self-serve physiological measurement device, according to one embodiment of the invention;

FIG. 4A shows a block diagram which implements at least one of a biometric registration device, biometric check-in device, and physiological measurement device alone or in combination, according to one embodiment of invention;

FIG. 4B depicts a kiosk which is implemented with at least one of a biometric registration device, biometric check-in device, and physiological measurement device alone or in combination, according to one embodiment of invention;

FIG. 4C depicts a surface-mounted device which is implemented with at least one of a biometric check-in device, and physiological measurement device alone or in combination, according to one embodiment of invention;

FIG. 4D depicts a scale which is implemented with at least one of a biometric check-in device, and physiological measurement device alone or in combination, according to one embodiment of invention;

FIG. 4E depicts a handheld device which is implemented with at least one of a biometric check-in device, and physiological measurement device alone or in combination, according to one embodiment of invention; and

FIG. 5 shows a block diagram of the data structure of a data store, according to one embodiment of the invention.

### DETAILED DESCRIPTION

Reference will now be made in detail to an implementation consistent with the present invention as illustrated in the accompanying drawings. Wherever possible, the same reference numbers will be used throughout the drawings and the following description to refer to the same or like parts. Although discussed with reference to these illustrations, the present invention is not limited to the illustrations therein. Hence, the reader should regard these illustrations merely as examples of embodiments of the present invention, the full scope of which is measured only in terms of the claims following this description.

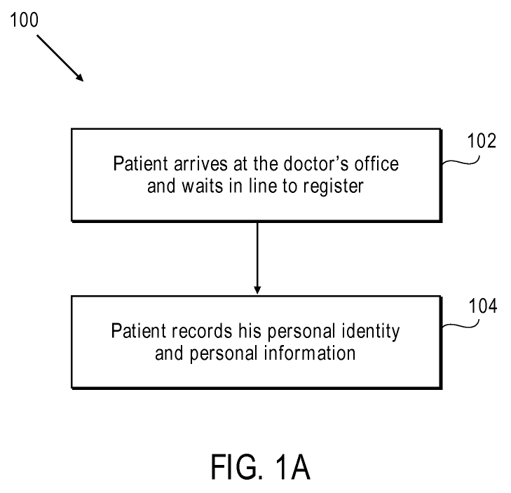

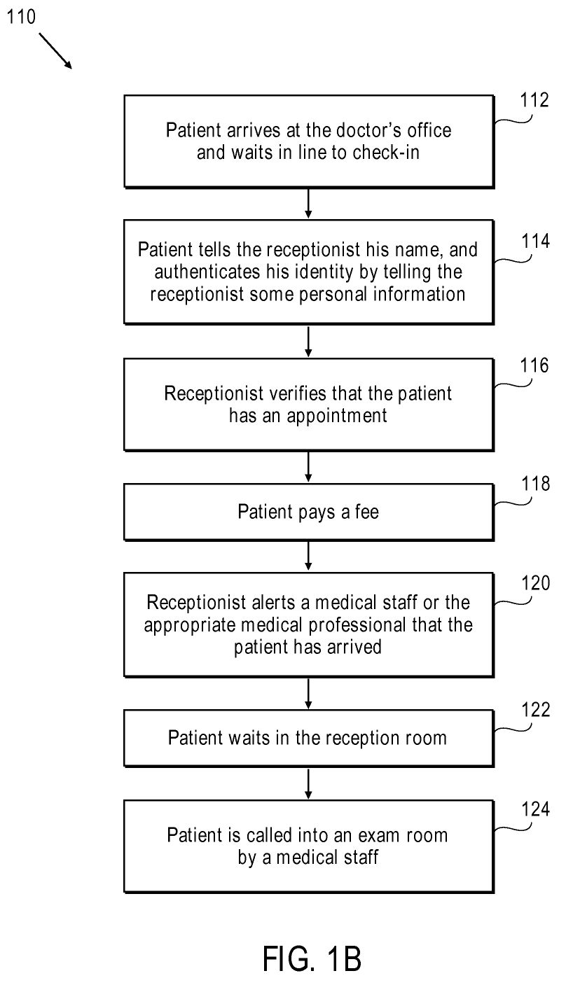

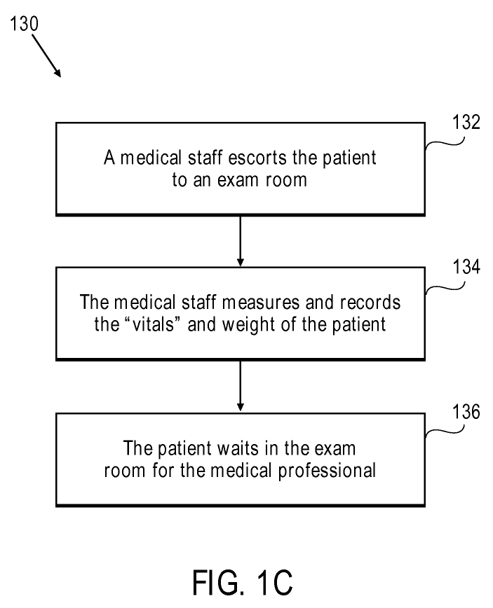

FIG. 1A shows a flow diagram of a conventional patient registration process 100, which takes place during a patient's first visit to consult a medical professional. A patient arrives at the reception area of the doctor's office, and must wait in line for a receptionist to assist him with the registration process (step 102). When assisted, a patient typically is requested to complete forms to record his personal identity and personal information. Personal identity includes, but is not limited to a patient's name and Social Security number. Personal information includes, but is not limited to a patient's Social Security number, date of birth, residence address, marital status, emergency contact information, phone number, and health insurance information. This personal information is often used to authenticate a patient's identity, during subsequent visits to the doctor's office. Upon completing the registration process, the patient proceeds to step 116 of the patient check-in procedure depicted in FIG. 1B, in which a receptionist verifies that the patient has an appointment.

FIG. 1B shows a flow diagram of a conventional patient check-in process 110, which takes place during a patient's subsequent visit to consult a medical professional. A patient arrives at the reception area of the doctor's office, and must wait for a receptionist to assist him with the check-in process (step 112). When assisted, a patient tells the receptionist his name, and is required to authenticate his identity by telling the receptionist some portions of his personal information, which might include his date of birth and phone number (step 114). The receptionist then verifies that the patient has an appointment to see the medical professional (step 116). The patient is requested to pay a fee for the appointment (step 118), typically called a co-payment, if the patient has health insurance. The receptionist alerts a medical staff member or the appropriate medical professional that the patient has arrived (step 120). Next, the patient waits in the reception room (step 122), until the patient is called into an exam room by a medical staff member (step 124). This completes the patient check-in process 110.

FIG. 1C shows a flow diagram of a conventional physiological measurement process 130, which typically takes place after the patient check-in process 110. A medical staff member escorts the patient to an exam room (step 132), and measures and records the vitals and weight of the patient (step 134). Vitals typically include heart rate, blood pressure, and body temperature. Lastly, the patient waits in an exam room for the medical professional (step 136).

In addition to the deficiencies mentioned in the background section, there are other flaws associated with the conventional patient intake and physiological measurement process. It should be now readily apparent that a typical doctor's office requires at least one receptionist fully devoted to the patient intake process, and one medical staff member fully devoted to the physiological measurement process. With a self-serve medical intake and physiological measurement process, as will be detailed shortly, receptionists and medical staff members can focus their attention on other tasks, such as fielding phone calls from ill patients and ordering medical tests. In one embodiment of the invention, the number of receptionists and medical staff members needed in a medical facility is greatly reduced, decreasing staffing costs.

A conventional doctor's office also requires a reception area, and a plurality of exam rooms for patients to wait for their physician after their physiological measurements have been taken by the medical staff. In one embodiment of the invention, the number of exam rooms can be reduced, decreasing facility costs.

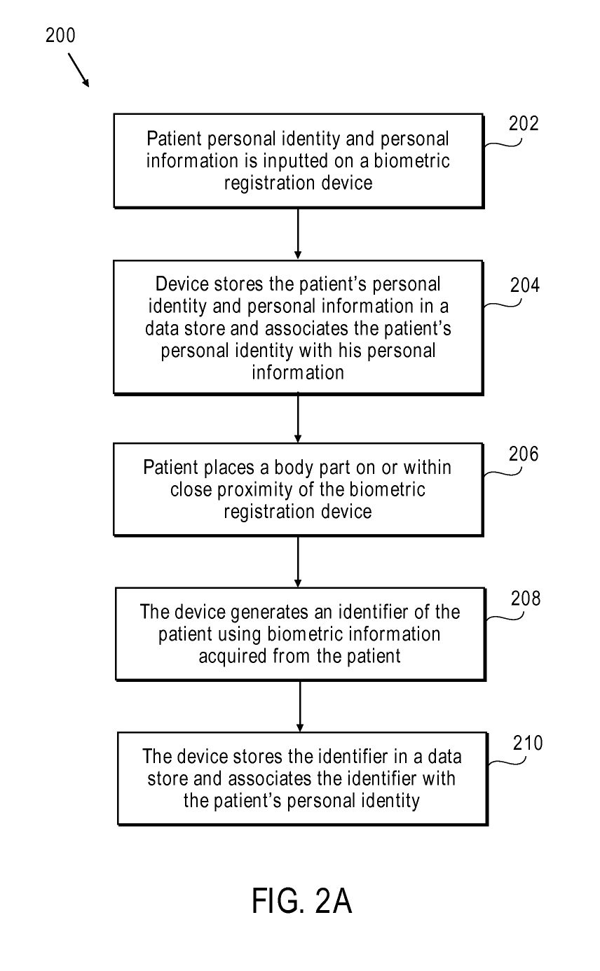

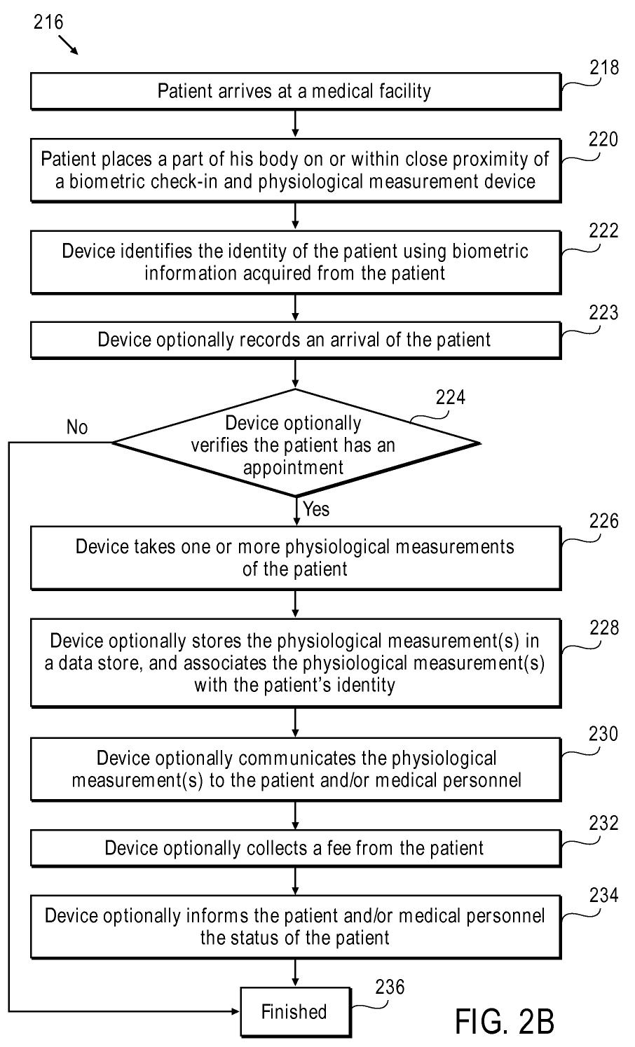

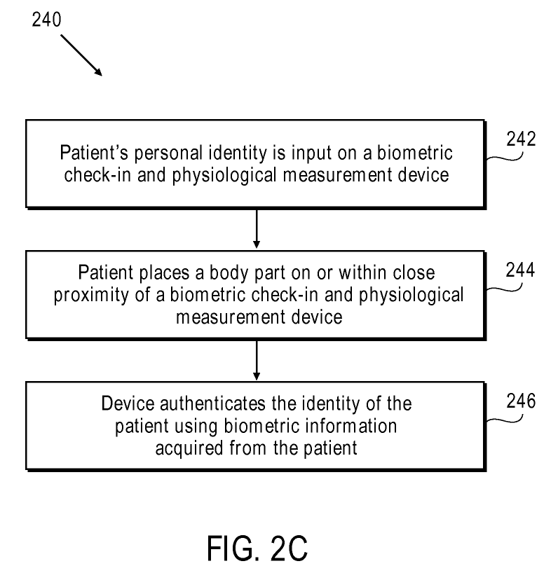

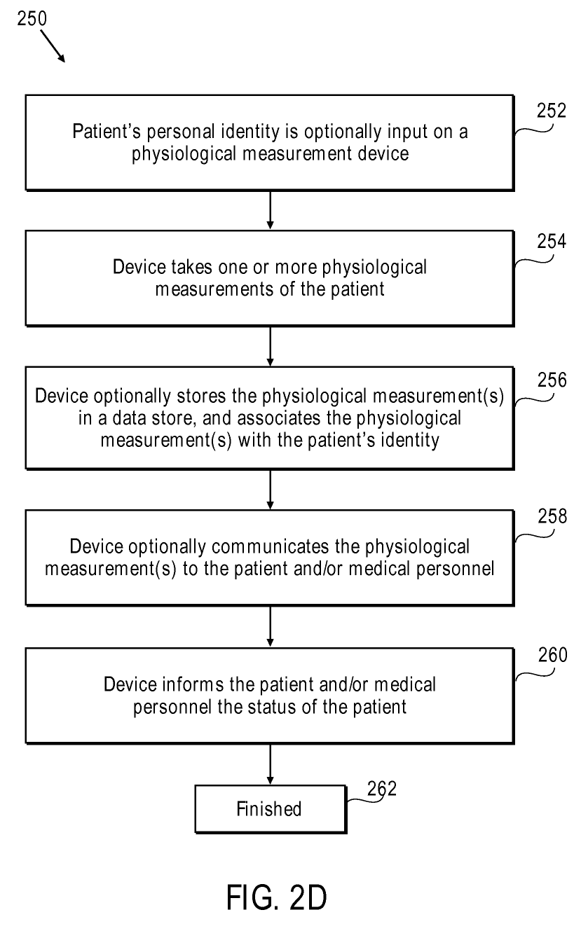

FIG. 2A shows a flow diagram of a biometric registration process 200, according to one embodiment of the invention. Biometric information is any quantitative measurement or characteristic of an individual, that can help to identify an individual. Common biometrics include quantitative measurements or characteristics that are unique to an individual, or at the very least are shared by very few individuals. Biometric information includes, but is not limited to fingerprints, DNA sequences, palm vein structure, blood vessel structure, iris patterns, voice patterns, facial features, and combinations thereof. Biometric information also includes quantitative measurements associated with bodily fluids including blood, urine, saliva, and combinations thereof. A biometric sensor is a sensor that measures biometric information.

In the patient biometric registration process 200, a biometric registration device is used to store the patient's personal identity and personal information, along with acquiring biometric information of the patient, upon the patient's first visit to see the medical professional, according to one embodiment of the invention. First, the patient's personal identity and personal information is input on a biometric registration device (step 202). The biometric registration device may or may not be self-serve. For a self-serve biometric registration device, the patient or an assistant of the patient inputs the patient's personal identity and personal information. In a non-self-serve biometric registration device, the patient writes down or otherwise communicates his personal identity and personal information to a receptionist or medical staff member, who then transcribes the personal identity and personal information of the patient to electronic form. The biometric registration device stores the patient's personal identity and personal information in a data store and associates the patient's personal identity with his personal information (step 204). Next, the patient places a part of his body on or within close proximity of the biometric registration device (step 206). The device generates an identifier of the patient using biometric information acquired from the patient (step 208). The device stores the identifier in a data store and associates the identifier with the patient's personal identity (step 210). The patient then proceeds to step 223 of FIG. 2B, during which a biometric check-in and physiological measurement device records an arrival of the patient.

In a variation of the process depicted in FIG. 2A, the patient first places a body part on or within close proximity of the biometric registration device (step 206). Upon not recognizing the biometric information of the patient, the biometric registration device then prompts the patient to enter his personal identity and personal information. The process then proceeds according to FIG. 2A, with step 206 omitted, and step 204 followed by step 208.

FIG. 2B shows a flow diagram of a biometric check-in and physiological measurement process 216, which occurs during a patient's subsequent visit to the medical facility, according to one embodiment of the invention. After arriving at a medical facility (step 218), a patient places a part of his body on or within close proximity of a biometric check-in and physiological measurement device (step 220). The device acquires biometric information from the patient, and generates an identifier from the biometric information following the same procedure used to generate the identifier during the biometric registration process (step 208). Using the identifier, the device retrieves the personal identity corresponding to the identifier, completing step 222. When used to identify a patient, the biometric information must be unique to the patient, or at the very least be shared by very few individuals, so as to minimize the probability of incorrectly identifying the patient.

In an alternate mode of operation, steps 220 and 222 may be replaced by the flow diagram depicted in FIG. 2C. In this mode of operation, biometric information is used to authenticate the identity of the patient, rather than to identify the patient. First, the patient's personal identity is input on a biometric check-in and physiological measurement device (step 242) through a keyboard, microphone, a touch screen device, or a card reader, to name a few of the many possible input mechanisms of the device. The patient's personal identity may be input by the patient, an assistant of the patient, receptionist, or medical staff member. The patient then places a part of his body on or within close proximity of a biometric check-in and physiological measurement device (step 244). The device acquires biometric information from the patient, and generates an identifier from the biometric information following the same process used to generate the identifier during the biometric registration process (step 208). Using the identifier, the device retrieves the personal identity corresponding to the identifier from a data store, and compares the retrieved personal identity with the personal identity input on the device. If the retrieved personal identity matches the input personal identity, step 246 is complete, and the process proceeds to step 223 of flow diagram 216.

The device optionally records an arrival of the patient (step 223) and then optionally verifies that the patient has an appointment with a medical professional (step 224). If the patient does not have an appointment, the process terminates or raises a notice condition to alert the receptionist or other member of the office to the unscheduled visit. Otherwise, the process continues to step 226, during which the device non-invasively takes one or more physiological measurements of the patient. In particular, a non-invasive measurement does not penetrate the skin of the patient. In comparison with the conventional physiological measurement process which requires the assistance of the medical staff, the present invention eliminates the need for medical staff assistance during the physiological measurement process. The device then optionally stores the physiological measurements(s) in a data store, and associates the physiological measurement(s) with the patient's identity (step 228). The device optionally communicates the physiological measurement(s) to the patient and/or medical personnel 230. Medical personnel include, but are not limited to a medical staff member and a medical professional. The device may communicate the physiological measurement(s) to the patient and/or medical personnel using output mechanisms including, but not limited to a printer, display, and speaker.

The device then optionally collects a fee for the patient, either by check, cash, or credit/debit card (step 232). Note, the patient may not have to pay a fee at all, or may be requested to pay a fee after the appointment with the medical professional. Finally, the device optionally informs the patient and/or the medical personnel the status of the patient. The patient status may include, but is not limited to “patient is ready to be examined,” “patient is not ready to be examined,” “check-in completed,” and “check-in not completed.” The patient may be informed of the status through a speaker, printer, and display, to name a few examples. The medical personnel may be informed of the status through a speaker, an intercom system, medical staff member, a pager, a printer, and a display, to name a few examples. A medical personnel may also be informed of the status through a light outside the exam room, where the presence, absence, intensity, or color of the light indicates a status of the patient. At the conclusion of step 234, the biometric check-in and physiological measurement process terminates.

In another embodiment of the invention, the device does not verify that the patient has an appointment, with step 224 eliminated from flow diagram 216, and step 223 proceeding directly to step 226. This altered version of the check-in process might occur when a patient goes to the hospital to treat an urgent medical condition, as patients typically do not schedule appointments in advance for hospital visits. In this embodiment of the invention, steps 220, 222, 223, 226, 228, 230, and 232 may take place under the supervision of a medical professional. Further, step 234 is additionally eliminated in flow diagram 216, as there is no longer the need to inform the patient and/or medical professional the status of the check-in, as the medical professional is already supervising the care of the patient.

In steps 208 and 222, the biometric information includes, but is not limited to fingerprints, DNA sequences, palm vein structure, blood vessel structure, iris patterns, voice patterns, facial features, and combinations thereof. Biometric information also includes quantitative measurements associated with bodily fluids including blood, urine, saliva, and combinations thereof Biometric information can be acquired from the patient's skin, hand, foot, eye, or any part of the patient.

A physiological measurement involves measuring a physiological characteristic of a patient which includes, but is not limited to weight, percent body fat, blood pressure, heart rate, oxygen level, body temperature, and optical refraction. In one embodiment of the invention, several physiological measurements can be taken with one scan of the patient's body part. In another embodiment of the invention, a patient's blood pressure may be measured through the patient's blood vessel structure using a biometric sensor which is cuffless. Cuffless blood pressure measurement is well known and is described in U.S. Pat. No. 7,179,228, herein incorporated by reference. The patient may place his hand or foot on or over the biometric sensor, avoiding the discomfort of a cuff inflated around his arm. Numerous sources of measurement inaccuracy are avoided including improper detection of pulse and improper placement of the cuff.

In one embodiment of the invention, the physiological characteristic is measured from the same part of the human body from which biometric information is acquired for the identification or authentication of the identity of the patient. For example, a scan of the hand may measure a physiological characteristic as well as acquire biometric information for the identification or authentication of the identity of the patient. In particular, information from blood vessels may be used to determine blood pressure, as well as identify or authenticate the patient. Similarly, a scan of the foot may measure a physiological characteristic as well as acquire biometric information for the identification or authentication of the identity of the patient. In particular, a scan of the foot may measure the percent of body fat in the patient, as well as acquire biometric information, such as blood vessel structure, for the identification or authentication of the patient. Another example involves a visit to the optometrist office. Scanning the eyes can generate a physiological measurement, such as the optical refraction of the eye, as well as an image of the iris which can be used for identification or authentication of the identity of the patient.

FIG. 2D shows a flow diagram 250 of a self-serve physiological measurement process, according to one embodiment of the invention. This physiological measurement process may be, but is not necessarily performed after a biometric check-in process, or after a conventional check-in process which requires the assistance of a receptionist. The patient's personal identity is optionally input on a physiological measurement device (step 252). The device non-invasively takes one or more physiological measurements of the patient (step 254). In comparison with the conventional physiological measurement process which requires the assistance of the medical staff, the present invention eliminates the need for medical staff assistance during the physiological measurement process. The device then optionally stores the physiological measurements(s) in a data store, and associates the physiological measurement(s) with the patient's identity (step 256). The device optionally communicates the physiological measurement(s) to the patient and/or medical personnel 258. The device may communicate the physiological measurement(s) to the patient and/or medical personnel using output mechanisms including, but not limited to a printer, display, and speaker.

Finally, the device optionally informs the patient and/or the medical personnel the status of the patient. The status may include, but is not limited to “patient is ready to be examined,” “patient is not ready to be examined,” “physiological measurements completed,” and “physiological measurements not completed.” The patient may be informed of the status through a speaker, printer, and display, to name a few examples. The medical personnel may be informed of the status through a speaker, an intercom system, medical staff member, a pager, a printer, and a display, to name a few examples. A medical personnel may also be informed of the status through a light outside the exam room, where the presence, absence, intensity, or color of the light indicates a status of the patient. At the conclusion of step 260, the physiological measurement process terminates.

A physiological measurement involves measuring a physiological characteristic of a patient which includes, but is not limited to weight, percent body fat, blood pressure, heart rate, oxygen level, body temperature, and optical refraction. The physiological measurement may, but does not necessarily use a biometric sensor. In particular, the physiological measurement may use conventional physiological measurement equipment, such as, but not limited to, a thermometer to measure temperature, or a sphygmomanometer to measure blood pressure.

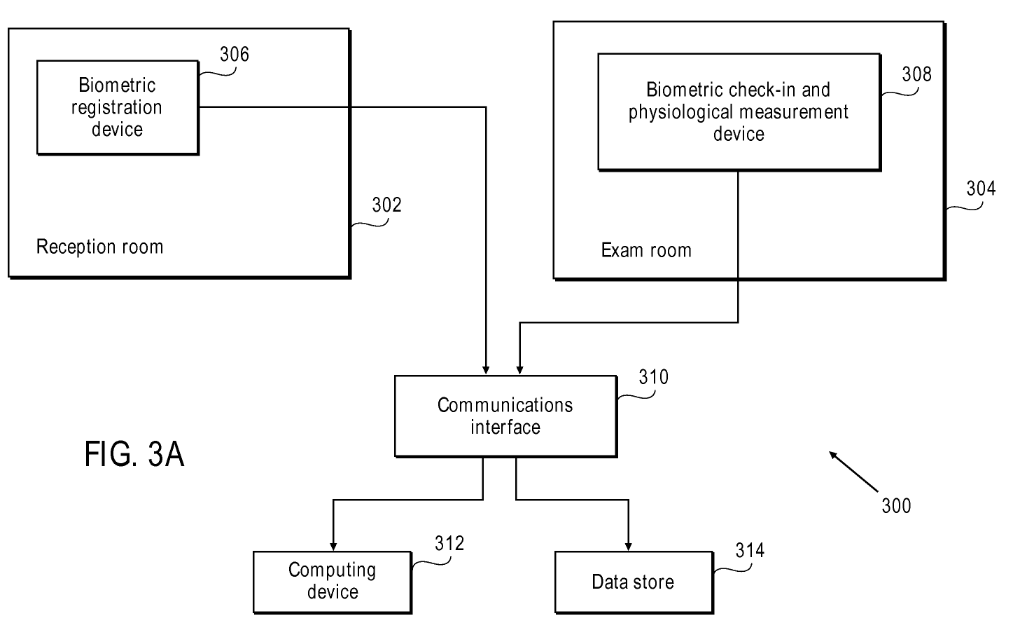

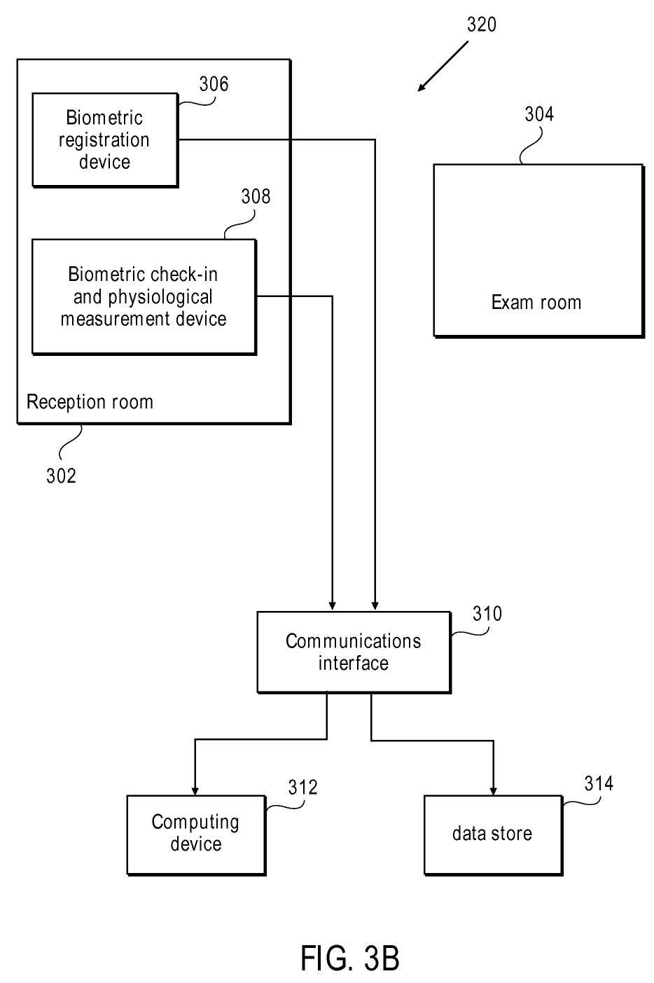

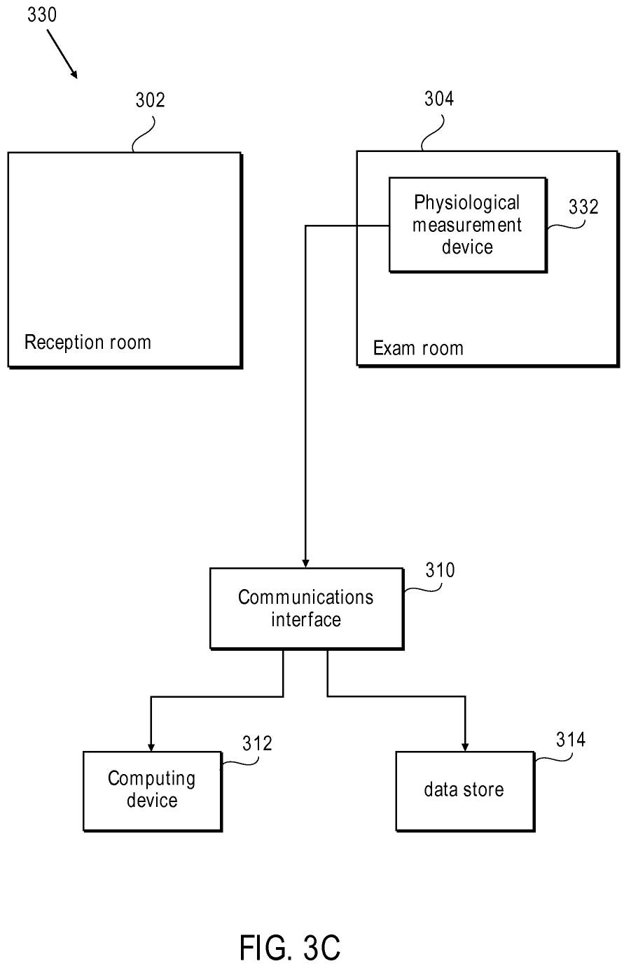

FIG. 3A shows a block diagram 300 of a medical facility configured for biometric registration, biometric check-in, and physiological measurement, according to one embodiment of the invention. The medical facility includes a reception room 302, and one (or more) exam rooms 304. Located in reception room 302 is a biometric registration device, and located in or near exam room 304 is a biometric check-in and physiological measurement device 308. Computing device 312 and data store 314 may be located in the medical facility, or in a site external to the medical facility. The computing device 312 includes, but is not limited to a desktop computer, laptop, workstation, tablet computer, server, handheld device, and mainframe. The data store 314 includes, but is not limited to RAM (Random Access Memory), a hard drive, a floppy disk, EEPROM (Electrically Erasable Programmable Read-Only Memory), flash memory, and cache. The biometric registration device 306, biometric check-in and physiological measurement device 308, computing device 312, and data store 314 are interfaced by a communications interface 310. The communications interface 310 includes, but is not limited to a Local Area Network, Wireless Network, Wide Area Network, Metropolitan Area Network, data bus, USB (Universal Serial Bus), SPI (Serial Peripheral Interface), and I²C (Inter-Integrated Circuit). It is understood that the data store 314 may be a part of the biometric registration device 306, the biometric check-in and physiological measurement device 308, or the computing device 312. The data store 314 may also be a stand-alone unit, such as a flash drive, memory card, CD-RW (Compact Disc ReWritable), and DVD-RW (Digital Video Disc ReWritable), to name a few examples. It is also understood that the computing device 312 may be a part of the biometric registration device 306, and the biometric check-in and physiological measurement device 308.

In one embodiment of the invention, the patient interacts with the biometric registration device 306 located in the reception room 302, during an initial visit to consult the medical professional. Upon subsequent visits to consult the medical professional, the patient waits in the reception room 302, and does not interact with any device. Upon the availability of an exam room 304, the patient proceeds to an exam room 304, and interacts with a biometric check-in and physiological measurement device 308. A medical staff or receptionist may inform the patient of an available exam room 304. Alternatively, an electronic system may inform a patient of an available exam room 304. For example, the exam rooms 304 may be numbered, and an electronic surveillance system may monitor whether an exam room 304 is occupied or not. In the reception room 302, a display displays the numbers of the available exam rooms 304. At the conclusion of the check-in and physiological measurement process, the medical professional is alerted, and the medical professional treats the patient. Indeed, there is the need for very few receptionists and medical staff, if any at all. In one embodiment, there are no receptionists or medical staff.

FIG. 3B shows a block diagram 320 of a medical facility configured for biometric registration, biometric check-in, and physiological measurement, according to one embodiment of the invention. In this embodiment, there is preferably only one reception room 302, and one exam room 304 for each medical professional. The biometric registration device 306 and biometric check-in and physiological measurement device 308 are located in the reception room 302. The computing device 312 and data store 314 are once again either located in the medical facility, or are located in a site external to the medical facility. The biometric registration device 306, biometric check-in and physiological measurement device 308, computing device 312, and data store 314 are interfaced by a communications interface 310.

In one embodiment of the invention, the patient interacts with a biometric registration device 306, followed by interacting with a biometric check-in and physiological measurement device 308, during an initial visit to consult a medical professional. The patient only interacts with the biometric check-in and physiological measurement device 308 during subsequent visits to consult a medical professional. Upon the availability of the medical professional, and in response to the medical professional being alerted that a patient has completed the biometric check-in and physiological measurement process, the medical professional escorts the patient from the reception room 302 into the exam room 304. Indeed, the medical facility may have only one reception room 302, and one exam room 304 for each medical professional, as there is no longer the need for patients to wait in exam rooms after their physiological measurements have been taken. Accordingly, there is also no need to alert patients of the availability of an exam room in this embodiment of the invention. Once again, there is the need for very few receptionists and medical staff, if any at all. In one embodiment, there are no receptionists or medical staff.

FIG. 3C shows a block diagram 330 of a medical facility configured with a self-serve physiological measurement device, according to one embodiment of the invention. After checking-in to the medical facility, the patient proceeds into an exam room 304, and uses the physiological measurement device 332, to measure one or more physiological characteristics without the assistance of medical staff. The physiological measurement device 332, computing device 312, and data store 314 are interfaced by a communications interface 310. Once again, it is understood that the computing device 312 and/or data store 314 may be a part of the physiological measurement device 332.

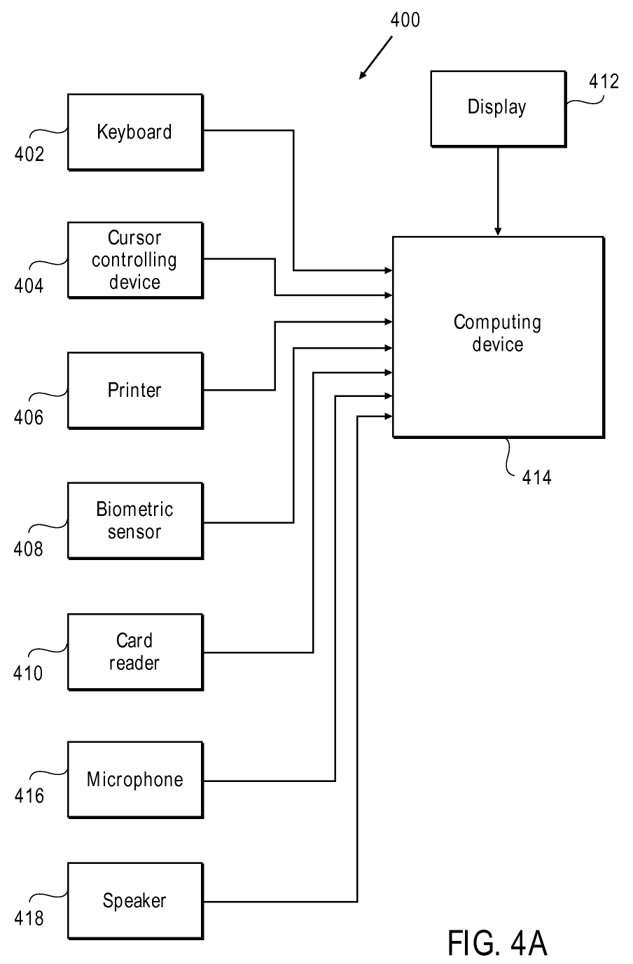

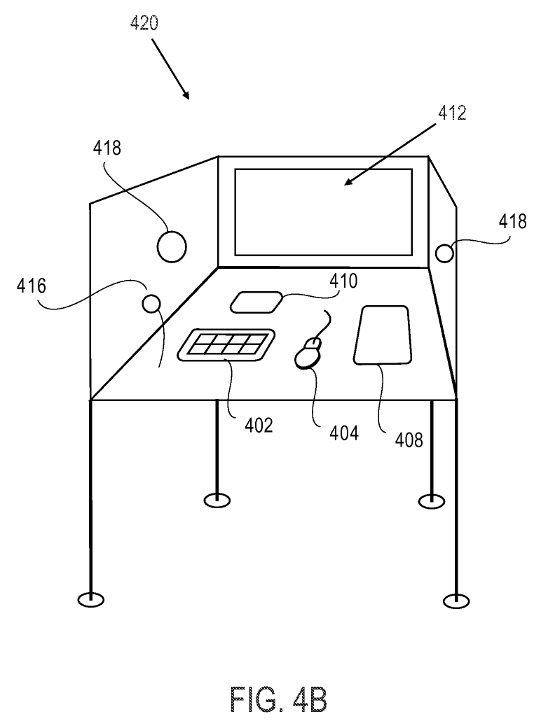

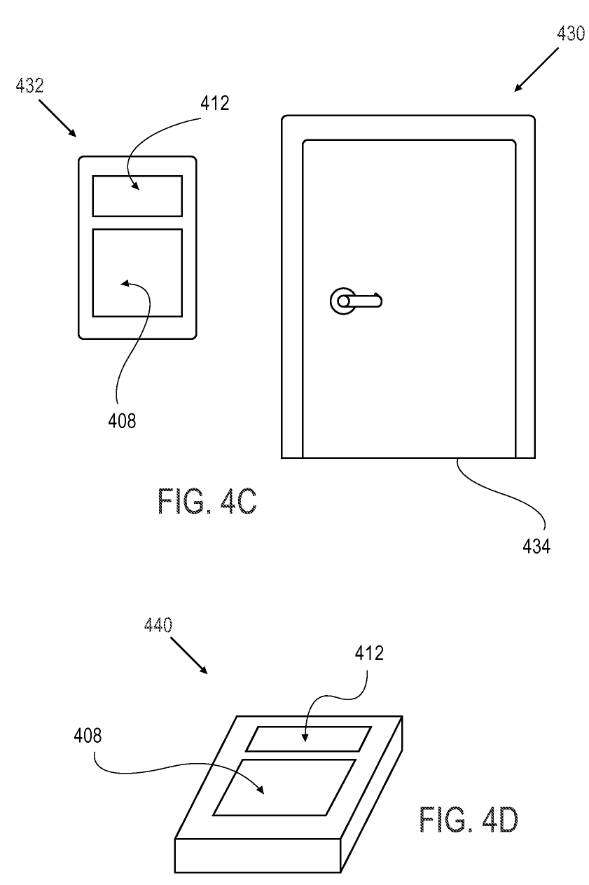

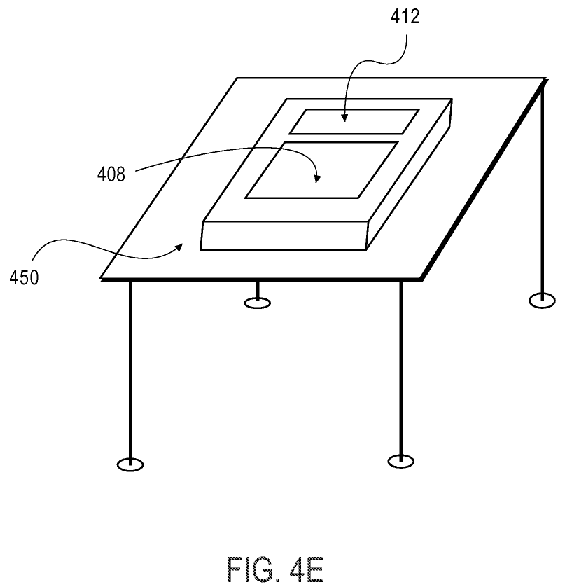

It is noted that the biometric registration device, and biometric check-in and physiological measurement device may be implemented as two separate devices, or incorporated together into a single device. FIG. 4A shows a block diagram 400, which implements at least one of a biometric registration device, biometric check-in device, and physiological measurement device alone or in combination, according to one embodiment of invention. The device 400 optionally includes a keyboard 402, cursor controlling device 404, printer 406, biometric sensor 408, card reader 410, microphone 416, speaker 418, and display 412 interfaced with a computing device 414. A cursor controlling device 404 includes, but is not limited to a computer mouse, joy stick, game controller, touchpad, and pen and tablet. The card reader 410 includes, but is not limited to a credit card reader and memory card receptacle.

An interface between components may be a direct or indirect interface, where the components include 402, 404, 406, 408, 410, 412, 414, 416, and 418. A direct interface means two components are interfaced with one another without the interface involving other components. An indirect interface means two components are interfaced with one another through another component. For example, the keyboard 402 is directly interfaced with the computing device 414. In another embodiment, the cursor controlling device 404 may be indirectly interfaced with the computing device 414 through a USB port on keyboard 402.

Keyboard 402, cursor controlling device 404, display 412, and microphone 416 may be used by the patient to input their personal identity and personal information during the biometric registration, and biometric check-in and physiological measurement processes. Printer 406, speaker 418, and display 412 may be used by the computing device 414 to inform the person of the status of the check-in, as well as the physiological measurement results. Biometric sensor 408 may be used by the patient to input biometric information to the computing device 414. Card reader 410 may be used by the patient to input his personal identification, as well as pay a co-payment.

FIG. 4B depicts a kiosk 420, which is implemented with at least one of a biometric registration device, biometric check-in device, and physiological measurement device alone or in combination, according to one embodiment of invention. FIG. 4C depicts a surface-mounted device 432, which is implemented with at least one of a biometric check-in device, and physiological measurement device alone or in combination, according to one embodiment of invention. More specifically, surface-mounted device 432 may be a wall-mounted device. In one embodiment of the invention, door 434 of the exam room is locked until the patient's identity is successfully authenticated by device 432, and the device has verified that the patient has an appointment. This surface-mounted device is especially useful for the medical facility described in the FIG. 3A, where component 308 may be a surface-mounted device. Surface-mounted device 432 prevents unauthorized persons from occupying an available exam room, and assures the medical professional that only a patient with an appointment is waiting in an exam room when the medical professional enters the exam room.

The surface mounted device 432 includes biometric sensor 408, and display 412. In another embodiment, surface mounted device 432 only includes a biometric sensor 408. Alternatively, device 432 may be mounted on the medical facility building door.

In another embodiment of the invention, a door knob with a biometric sensor built into the door knob may serve a similar function as surface-mounted device 432. By simply grabbing the biometric door knob of the exam room, the patient is immediately checked-in; the patient's physiological measurements are taken; and the patient is ready to be seen by the medical professional. Similarly, the exam room is locked until the patient's identity is successfully authenticated by the biometric door knob, and the door knob has verified that the patient has an appointment. Alternatively, the biometric door knob may be installed on the medical facility building door.

FIG. 4D depicts a scale 440, which is implemented with at least one of a biometric check-in device, and physiological measurement device alone or in combination, according to one embodiment of invention. The scale has a biometric sensor 408 and display 412. This embodiment of the invention is especially suitable to measure a patient's weight and percent body fat. FIG. 4E depicts a handheld device 450, which is implemented with at least one of a biometric check-in device, and physiological measurement device alone or in combination, according to one embodiment of invention. The handheld device has a biometric sensor 408 and display 412. The handheld device 450 may be placed on a bed, counter, or table in the reception or exam room.

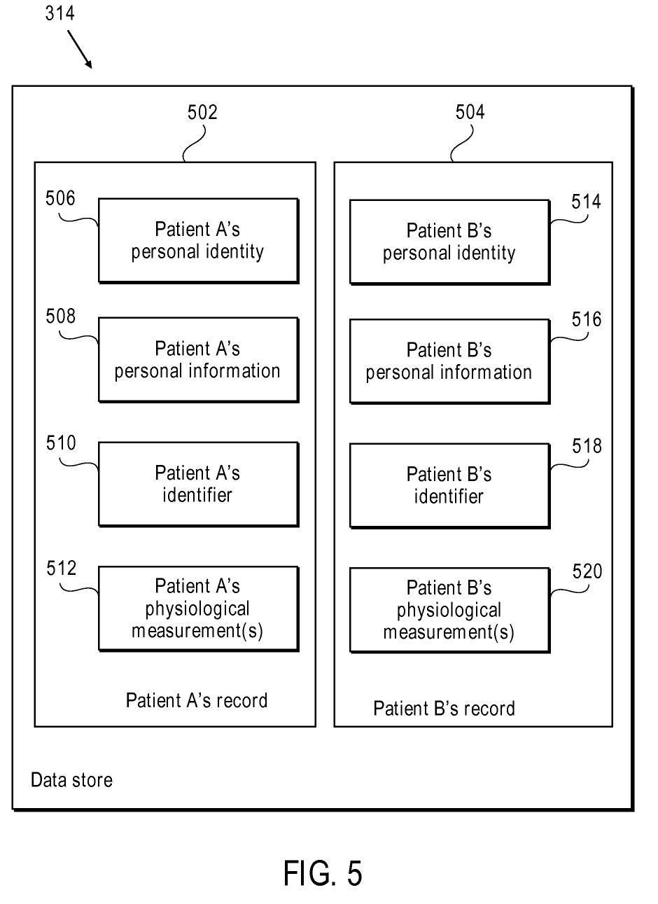

FIG. 5 shows a block diagram of the data structure of a data store, according to one embodiment of the invention. In one embodiment, data store 314 stores one or more patient records. Two patient records, patient A's record 502, and patient B's record 504 are depicted in data store 314, as examples. In a patient record, a patient's personal identity, personal information, identifier, and physiological measurement(s) are associated together. Patient A's personal identity 506, personal information 508, identifier constructed from biometric information 510, and physiological measurement(s) 512 are associated together in patient A's record 502. Similarly, Patient B's personal identity 514, personal information 516, identifier, and physiological measurement(s) 520 are associated together in patient B's record 504.

The present invention provides patients with a convenient and comfortable intake and physiological measurement process, through processing biometric information acquired from the patient, as well as advanced physiological measurement techniques. With cuffless blood pressure measurements, a patient experiences less discomfort with blood pressure measurement than conventional methods of blood pressure measurement. The patient no longer needs to orally recite their personal information to a receptionist, and risk their personal information being misused by an eavesdropper. In addition, the number of receptionists, medical staff, and exam rooms can be reduced in a medical facility, thereby reducing staffing and facility costs.

The scope of the invention is defined by the following claims and their equivalents.

What is claimed is:

1. A method, comprising facilitating a patient's arrival at a medical facility through the patient's interaction with a biometric check-in and physiological measurement device, wherein the device identifies the patient or authenticates an identity of the patient using biometric information acquired from the patient, measures a physiological characteristic of the patient, and performs at least one of recording an arrival of the patient at the medical facility or verifying the patient has an appointment.

2. The method of claim 1, wherein the physiological characteristic is measured from a body part of the patient from which biometric information is acquired for the identification or authentication of the identity of the patient.

3. The method of claim 1, wherein the biometric information has been acquired from a hand of the patient.

4. The method of claim 1, wherein the biometric information has been acquired from a foot of the patient.

5. The method of claim 1, wherein the device identifies the patient or authenticates the identity of the patient using biometric information acquired from skin of the patient.

6. The method of claim 1, wherein the device identifies the patient or authenticates the identity of the patient using biometric information acquired from a blood vessel of the patient.

7. The method of claim 1, wherein the physiological characteristic is weight.

8. The method of claim 1, wherein the physiological characteristic is percent body fat.

9. The method of claim 1, wherein the physiological characteristic is blood pressure.

10. The method of claim 9, wherein the blood pressure is measured without a cuff.

11. The method of claim 1, wherein the physiological characteristic is heart rate.

12. The method of claim 1, wherein the physiological characteristic is oxygen level.

13. The method of claim 1, wherein the physiological characteristic is body temperature.

14. The method of claim 1, wherein the device non-invasively measures a physiological characteristic of the patient.

15. The method of claim 1, further comprising the patient paying a fee.

16. The method of claim 1, further comprising informing the patient or a medical personnel of a status of the patient.

17. The method of claim 1, further comprising informing the patient or a medical personnel of a physiological measurement of the patient.

18. The method of claim 1, further comprising storing a physiological measurement in a data store.

19. A biometric check-in and physiological measurement device, comprising means for identifying a patient or authenticating an identity of the patient using biometric information acquired from the patient, means for recording an arrival of the patient at a medical facility or verifying the patient has an appointment, and means for measuring a physiological characteristic of the patient.

20. The device of claim 19, wherein the physiological characteristic is measured from a body part of the patient from which biometric information is acquired for the identification or authentication of the identity of the patient.

21. The device of claim 19, wherein the device is selected from the group consisting of a kiosk, handheld device, scale, door knob, and surface-mounted device.

22. The device of claim 21, wherein the surface-mounted device and door knob each controls a locking mechanism of an exam room door.

23. The device of claim 19, wherein the biometric information has been acquired from a hand of the patient.

24. The device of claim 19, wherein the biometric information has been acquired from a foot of the patient.

25. The device of claim 19, wherein the device identifies the patient or authenticates the identity of the patient using biometric information acquired from skin of the patient.

26. The device of claim 19, wherein the device identifies the patient or authenticates the identity of the patient using biometric information acquired from a blood vessel of the patient.

27. The device of claim 19, wherein the physiological characteristic is weight.

28. The device of claim 19, wherein the physiological characteristic is percent body fat.

29. The device of claim 19, wherein the physiological characteristic is blood pressure.

30. The device of claim 19, wherein the device is a cuffless device.

31. The device of claim 19, wherein the physiological characteristic is heart rate.

32. The device of claim 19, wherein the physiological characteristic is oxygen level.

33. The device of claim 19, wherein the physiological characteristic is body temperature.

34. The device of claim 19, wherein the means for measuring is non-invasive.

35. The device of claim 19, further comprising means to collect a fee.

36. The device of claim 19, further comprising means to inform the patient or a medical personnel a status of the patient.

37. The device of claim 19, further comprising means to inform the patient or a medical personnel a physiological measurement.

38. The device of claim 19, further comprising means to store a physiological measurement.

39. A method, comprising a physiological measurement device measuring a physiological characteristic of a patient without a medical staff member's assistance and informing the patient or a medical personnel of a status of the patient.

40. The method of claim 39, wherein the physiological characteristic is selected from the group consisting of weight, percent body fat, blood pressure, heart rate, oxygen level, body temperature, and optical refraction.

41. The method of claim 39, further comprising storing a physiological measurement in a data store.

42. The method of claim 39, further comprising informing the patient or a medical personnel of a physiological measurement of the patient.

43. A physiological measurement device, comprising means for measuring a physiological characteristic of the patient without a medical staff member's assistance and means for informing the patient or a medical personnel of a status of the patient.

44. The device of claim 43, wherein the physiological characteristic is selected from the group consisting of weight, percent body fat, blood pressure, heart rate, oxygen level, body temperature, and optical refraction.

45. The device of claim 43, further comprising means to store a physiological measurement.

46. The device of claim 43, further comprising means to inform the patient or a medical personnel a physiological measurement.
